(19) United States
(12) Patent Application Publication
(10) Pub. No.: US 2026/0151684 A1
(43) Pub. Date: Jun. 4, 2026

(54) GOLF SCORING AND ANALYSIS SYSTEM FOR PLAYER DEVELOPMENT

(71) Applicant: Patrick Ty, Redmond, WA (US)

(72) Inventor: Patrick Ty, Redmond, WA (US)

(21) Appl. No.: 18/964,708

(22) Filed: Dec. 2, 2024

(51) Int. Cl. A63B 71/06; A63B 24/00; A63B 102/32

(52) U.S. Cl. CPC A63B 71/0622; A63B 24/0062; A63B 71/0672; A63B 2102/32

(57) ABSTRACT

The present invention provides a comprehensive golf scoring and analysis system designed to enhance player development by capturing and analyzing detailed performance metrics. The system includes a specially designed paper scorecard for recording essential data on tee shots, approach shots, chip shots, putts, and penalties. The players or scorers use the scorecard to log shot details, which are then digitized through a mobile application using an AI-powered OCR engine. The data is processed in a cloud-based system and stored in an application database, enabling in-depth analysis of key metrics such as driving distance, accuracy, and putting performance. The AI engine generates personalized training recommendations based on identified strengths, weaknesses, and improvement areas. This integrated solution provides players with actionable insights, facilitating continuous improvement and promoting engagement through an intuitive and user-friendly scoring method.

### FIELD OF THE INVENTION

The present invention relates to the field of sports performance tracking systems, and specifically a comprehensive scoring and analysis system for golf that enhances player development by providing a structured approach to capturing, analyzing, and interpreting performance data. The present invention integrates traditional scorekeeping elements with advanced data analytics and intuitive user interfaces to address the needs of golfers seeking long-term improvement in their game. The present invention leverages both digital and physical components to create a seamless data capture experience, incorporating tools that encourage actionable insights into a player's strengths, weaknesses, improvement opportunities, and potential development threats, thereby enabling a comprehensive evaluation of golf performance.

### BACKGROUND

In recent years, golf has seen a surge in technological advancements aimed at improving player performance and enhancing the experience of the game. As a result, the use of digital tracking systems has become a popular tool for monitoring individual progress and providing statistical insights into gameplay. However, the existing solutions, predominantly traditional scorecards and mobile applications, present a range of limitations that hinder their efficacy in offering a complete picture of a player's development over time. Traditional scorecards, a staple on the course, provide a simple means of logging scores but are fundamentally limited by design. They lack the space, structure, and granularity necessary for capturing a comprehensive range of performance data. Critical information that could aid in skill development, such as shot accuracy, stroke efficiency, and areas of improvement, remains unrecorded. These details are essential for long-term player progress but are omitted due to the scorecards'static nature.

Mobile applications for golf stat tracking, while more robust than the scorecards, introduce new challenges. Many apps utilize GPS to record data and deliver various metrics, however, these apps often have unintuitive interfaces that complicate data entry and interfere with the natural flow of the game. Also, the requirement to frequently interact with a device can be disruptive, affecting a player's focus and rhythm. Additionally, the data captured by these applications, though detailed, often lacks actionable insight that a player can immediately use to improve specific areas of their game. In effect, these mobile solutions, despite their potential, have not managed to overcome the barriers of usability, player engagement, and data relevance. Furthermore, even when equipped with the sophisticated tools, player adoption remains a challenge. Golfers, especially those aiming for long-term improvement, need tools that seamlessly integrate into their routine without causing distractions or additional burdens. The time and effort required to enter or capture data can deter users, resulting in inconsistent data collection, reduced engagement, and ultimately, minimal benefit to player development. This hurdle in sustaining user adoption underscores the need for a system that is both comprehensive and user-friendly.

The present invention of a new golf scoring and analysis system for player development addresses these critical gaps. The disclosed system combines the strengths of traditional and digital tools while mitigating their weaknesses and provides players with meaningful insights into their strengths, weaknesses, and opportunities for improvement, ensuring that data is both easily accessible and actionable.

### BRIEF SUMMARY

The present invention discloses a golf scoring and analysis system for player development. The golf scoring and analysis system introduces a novel method for collecting and analyzing golf performance data. The system includes a specially designed paper scorecard, engineered to capture essential game metrics or data points. Unlike the traditional scorecards, this enhanced scorecard facilitates the collection of the essential data points such as the distance to the pin, ball lies and conditions, position relative to the hole, and ball flight characteristics. This careful design allows one or more players to efficiently record these detailed aspects of their play without disrupting their focus or pace on the course. Paired with analytical software, the scorecard enables a streamlined data analysis process, transforming recorded metrics into actionable insights tailored for player development. The software interprets the data to highlight specific areas of strength, identify potential weaknesses, and reveal opportunities for targeted improvement. This combination of the innovative scorecard and robust analytics may provide a significant advancement over the existing solutions in golf performance tracking, offering a practical and integrated approach to fostering continuous player growth and skill refinement.

In an embodiment, the golf scoring and analysis system for player development starts with a unique paper-based scorecard designed to capture key details of each round. The one or more players or a designated scorer fill in essential information such as hole distances and par values at the start, adding further data as the round progresses. After completing a round, the one or more players can take a photo of their scorecard with a mobile application running on their respective computing devices, initiating the data analysis process. The system may employ OCR (Optical Character Recognition) technology to digitize the handwritten entries. In some embodiments, the one or more players may be prompted to verify their entries if the handwriting is unclear or if the AI (Artificial Intelligence) system needs further training for accuracy. Once the data is validated, the backend software performs a detailed analysis, breaking down the player's performance with metrics like average driving distance, fairways hit, greens in regulation, and various strokes gained categories, including off-the-tee, approach, around-the-green, and putting. Additionally, it may capture data on putting starting positions, lines, and common misses. The integrated AI engine may then identify patterns, strengths, and weaknesses in the player's game, highlighting potential threats to their performance and suggesting specific drills or training exercises to help them capitalize on improvement opportunities. This combination of precise scorecard data capture and advanced AI-driven analysis provides a comprehensive feedback loop, supporting players in making informed adjustments and continuously refining their skills.

In a specific embodiment, the present invention discloses a golf scoring and analysis system for player development. The system includes a physical player scorecard configured to record hole-by-hole scoring data and key metrics related to a player's round of golf, the scorecard including sections for capturing information about distances, ball lies and conditions, shot outcomes, and putting characteristics. The system further includes a mobile application executable on a user computing device, the mobile application configured to capture an image of the scorecard and transmit the image for data extraction and analysis. The system further includes a cloud storage service configured to temporarily store the captured image of the scorecard. The system further includes an image extraction API, deployed on a cloud-based server, configured to receive the image from the cloud storage service and extract hole scoring data from the image. The system further includes an AI engine, comprising an OCR and image processing module, configured to process the image and convert the extracted data into a structured format within an application database. The system further includes the application database, comprising a NoSQL database for general data and a SQL database for scorecard data, the database configured to store player scoring data, analytics, and community interactions. Further, the AI engine may be configured to generate personalized insights and recommendations in real-time for training drills and skill improvement based on the player's performance metrics and extracted data, and the mobile application may be further configured to present analytics and performance metrics to the player in an interactive manner and display the personalized insights and recommendations.

In an embodiment, the scorecard may be configured to be printed or purchased by the player prior to play and includes pre-defined sections for recording data inducing at least distances to pin, ball location, shot flight trajectory, and putting characteristics for each hole. The scorecard further includes an area for capturing player notes or observations about the round, allowing for additional context to be added to the scorecard data.

In an embodiment, the mobile application may be further configured to prompt the player to validate the extracted data based on OCR accuracy and legibility of handwriting on the scorecard. In an embodiment, the cloud storage service may be further configured to organize scorecard images by unique identifiers corresponding to the player and scorecard reference, allowing for efficient retrieval and temporary storage prior to the data extraction. In an embodiment, the image extraction API may be implemented as a Python script deployed within a serverless computing environment to process the scorecard image and parse the extracted data into the application database. In an embodiment, the AI engine may also include a generative pre-trained transformer (GPT) vision API configured to extract the scoring data from the scorecard image by identifying and interpreting each data entry location on the scorecard. In an embodiment, the application database utilizes the NoSQL database for storing community posts, messages, and user preferences, and the SQL database for storing the structured scorecard data, each configured for optimized data retrieval and processing. In an embodiment, the mobile application may further include a charting and visualization solution that is integrated with a third-party visualization tool and is configured to enable the player to interact with the performance metrics and view analytical reports generated from the scorecard data. In an embodiment, the charting and visualization solution may be configured to display metrics such as average driving distance, fairways hit, greens in regulation, strokes gained in different aspects of play, and putting performance indicators.

In an embodiment, the AI engine may further include a machine learning model trained on historical golf scoring data to provide accurate and personalized insights and recommendations for the player's training and skill improvement. In an embodiment, the AI engine may be further configured to identify specific player strengths, weaknesses, and improvement opportunities based on the analyzed data and present this information to the player via the mobile application's user interface. In an embodiment, the AI engine is further configured to provide trend analysis of the player's performance over multiple rounds, identifying patterns and long-term improvements or areas requiring additional focus.

In an embodiment, the mobile application may further include a user interface for accessing drill practice logs, viewing historical performance metrics, and interacting with a community of other users. The mobile application may be further configured to allow the player to upload video or image files for review alongside the scoring data, storing these in the application database for comprehensive performance tracking. Further, the cloud storage service may be further configured to automatically delete the image of the scorecard from storage once the data extraction has been completed and verified, maintaining player privacy and storage efficiency. Further, the image extraction API may be configured to operate asynchronously and scales dynamically according to user demand, ensuring high availability and efficient processing for large volumes of scorecard images. The mobile application may be further configured to display a summary report after each round, highlighting key performance metrics and suggesting specific drills or practice routines tailored to the player's recent performance. The mobile application may be further configured to enable a scorer, other than the player, to complete and capture the scorecard data, supporting flexibility in score entry methods. Further, the application database may include a scoring history log that allows the player to access, review, and analyze data from previous rounds for tracking long-term progress.

The golf scoring and analysis system for player development presents significant advantages over traditional systems, centering around three key innovations: an optimized paper scorecard, AI-powered data extraction, and advanced analysis with personalized recommendations. Firstly, the optimized paper scorecard is crafted to systematically capture actionable data points essential for meaningful improvement, such as identifying a player's strengths, weaknesses, improvement opportunities, and threats. Its familiar tri-fold design ensures players can efficiently record scoring and performance details with minimal distraction, keeping their focus on the game rather than on cumbersome data entry. Secondly, the system employs AI-powered OCR technology, allowing players to upload an image of their completed scorecard through a mobile application, where the AI seamlessly extracts scoring data. This eliminates the need for manual data input, a common frustration with existing solutions, and ensures accuracy by prompting players to validate entries if necessary, enhancing the data's reliability over time as the AI learns from each entry. Further, the AI-driven analytical engine provides a detailed analysis of the player's performance across various metrics, such as strokes gained and putts per hole, transforming raw data into meaningful insights. Beyond basic statistics, the system offers personalized recommendations tailored to the player's specific needs, suggesting drills and training plans aimed at addressing weaknesses and leveraging areas of strength. These features collectively support long-term skill development and foster player engagement by making the data actionable and convenient, reducing entry barriers and creating a more immersive experience. This approach enables continuous improvement and ensures players receive tailored feedback that motivates and guides their journey toward their goals.

### BRIEF DESCRIPTION OF THE ACCOMPANYING DRAWINGS

The novel features which are believed to be characteristic of the present invention, as to its structure, organization, use, and method of operation, together with further objectives and advantages thereof, will be better understood from the following drawings in which a presently preferred embodiment of the invention will now be illustrated by way of example. It is expressly understood, however, that the drawings are for the purpose of illustration and description only and are not intended as a definition of the limits of the invention. Embodiments of this invention will now be described by way of example in association with the accompanying drawings in which:

FIG. 1 is a diagram that illustrates a system environment within which various embodiments of the present invention are practiced.

FIG. 2 is a diagram that illustrate a table defining key information related to golf and its scoring system, in accordance with an embodiment of the present invention.

FIGS. 3a-3c are diagrams that illustrate exemplary scorecards, in accordance with an embodiment of the present invention.

FIG. 4 is a diagram that illustrate a flowchart for the golf scoring and analysis system's method operation, in accordance with an embodiment of the present invention.

Further areas of applicability of the present invention will become apparent from the detailed description provided hereinafter. It should be understood that the detailed description of exemplary embodiments is intended for illustration purposes only and is, therefore, not intended to necessarily limit the scope of the invention.

### DETAILED DESCRIPTION

As used in the specification and claims, the singular forms “a”, “an”, and “the” may also include plural references. For example, the term “an article” may include a plurality of articles. Those with ordinary skill in the art will appreciate that the elements in the figures are illustrated for simplicity and clarity and are not necessarily drawn to scale. For example, the dimensions of some of the elements in the Figures may be exaggerated, relative to other elements, to improve the understanding of the present invention. There may be additional components described in the foregoing application that are not depicted on one of the described drawings. In the event such a component is described, but not depicted in a drawing, the absence of such a drawing should not be considered as an omission of such design from the specification.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. Unless stated otherwise, terms such as “first” and “second” are used to arbitrarily distinguish between the elements. Thus, these terms are not necessarily intended to indicate temporal or other prioritization of such elements. While various exemplary embodiments of the disclosed invention have been described below it should be understood that they have been presented for purposes of example only, not limitations. It is not exhaustive and does not limit the invention to the precise form disclosed. Modifications and variations are possible considering the above teachings or may be acquired from practicing of the invention, without departing from the breadth or scope.

The present invention will now be described with reference to the accompanying drawings which should be regarded as merely illustrative without restricting the scope and ambit of the present disclosure.

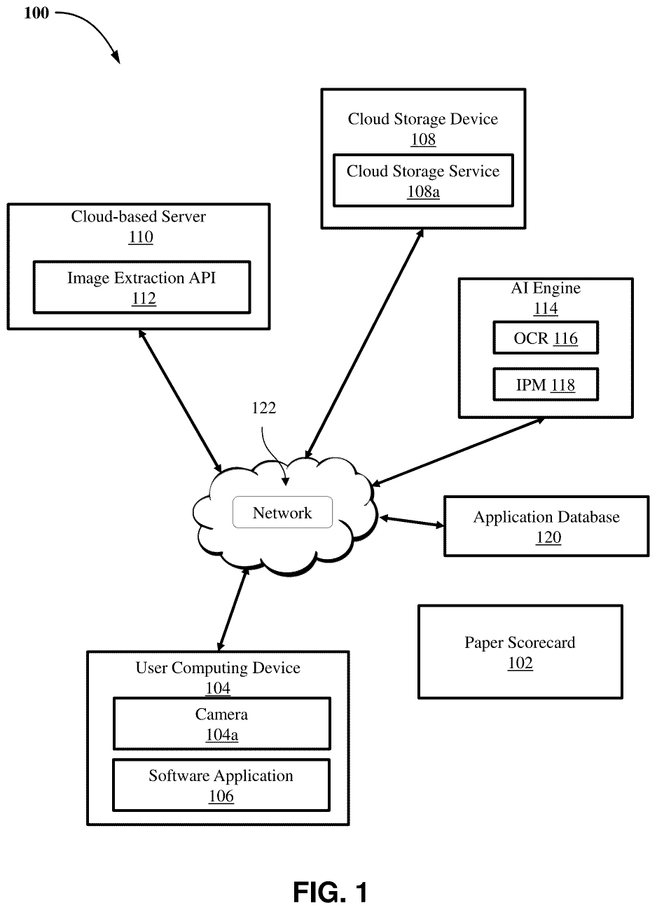

FIG. 1 is a diagram 100 that illustrates a system environment within which various embodiments of the present invention are practiced. The system environment includes a paper scorecard 102. The system environment further includes a user-computing device 104 including at least a camera 104a and a software or mobile application 106. The system environment further includes a cloud storage device 108 including a cloud storage service 108a. The system environment further includes a cloud-base server 110 including an image extraction API 112. The system environment further includes an AI engine 114 including an OCR module 116 and image processing module 118. The system environment further includes an application database 120. The user-computing device 104, the cloud storage device 108, the cloud-based server 110, the AI engine 114, and the application database 120 may be configured to communicate over a communication channel such as the network 122.

The paper scorecard 102 is a specially crafted tool designed to streamline the collection of detailed performance data for each hole in a round of golf. The scorecard 102 is tailored to capture a wide array of actionable metrics that reflect various aspects of the player's game. The scorecard 102 is made of paper or cardstock, and includes designated sections for recording essential information, such as distances to the pin, ball lies and conditions, shot outcomes (e.g., trajectory, over/short, left/right), and putting characteristics like distance, slope, and break direction. This detailed structure ensures that one or more players or their scorers can systematically document each shot, capturing a complete picture of the player's performance for later analysis. The scorecard's 102 design has been carefully crafted to align with traditional scorekeeping norms, ensuring ease of adoption while maintaining the flexibility needed for detailed data entry. With a folding layout, the scorecard 102 may offer the one or more players or scorers a user-friendly and efficient method of recording their game information. The scorecard 102 may be printed by the one or more players or scorers at home or purchased pre-printed, and its layout and spacing has been developed to facilitate seamless data extraction through AI-powered OCR technology, making it an integral part of the system's data processing workflow.

The user-computing device 104 refers to any mobile device, such as a smartphone or tablet or laptop, that the one or more players or their scorers may use to interface with the golf scoring and analysis system. The user-computing device 104 may be equipped with the camera 104a that helps in capturing a clear image of the filled-out paper scorecard 102 once the one or more rounds are complete. The camera 104a may enable the users (players or their scorers) to take high-resolution photos of the scorecard 102, which the system relies on for accurate data extraction. Alongside the camera 104a, the user-computing device 104 also runs a mobile application 106, which serves as the primary software interface for the players or their scorers. Through this mobile application 106, the users may capture and upload scorecard images, view personalized data insights, manage performance history, access drill practice logs, and interact with a community of fellow players. The mobile application 106 may be designed to facilitate seamless user interaction and ensure that scorecard data flows smoothly into the analysis pipeline. The user-computing device 102 may also include a processor that is configured to support the one or more operations of the mobile application 106 and handling tasks like image capture, storage, and initial processing. The processor also ensures that the data, including the scorecard image, can be managed, uploaded, and transmitted efficiently to the cloud for further analysis. Additionally, the processor enables the mobile application 106 to provide real-time feedback to the user, such as confirming successful uploads or displaying immediate summaries of past performance data. The processor may ensure smooth, responsive app functionality, allowing players to interact effortlessly with the scoring and analysis system.

The cloud storage device 108 refers to a robust and scalable storage infrastructure within the cloud that temporarily holds the one or more images of the scorecard 102 captured by the user-computing device 104. The cloud storage component is managed through a cloud storage service 108a, such as Google Cloud Storage or Amazon S3, which offers secure, reliable, and flexible data management in real-time. The cloud storage service 108 may help in the data workflow by providing a centralized location where the images can be stored temporarily after being uploaded from the user's mobile application 106. This temporary storage setup allows the system to efficiently manage and organize large volumes of data, particularly during periods of high user activity, by utilizing the scalable resources of cloud technology. The cloud storage service 108a may organize these images by assigning unique identifiers based on user-specific data, such as player ID or scorecard reference. This organization structure ensures that each scorecard image is accessible only to its respective user and is easy to retrieve for processing. Once the image is stored, the cloud storage service 108a may initiate the automated triggers that activate the next step in the data pipeline, specifically, the image extraction API 112. Furthermore, the cloud storage service 108a may be designed with security features to protect the sensitive player information, using encryption protocols to safeguard data during storage and transit. Once the image extraction and data validation processes are complete, the cloud storage device 108 may delete the scorecard image, adhering to the data minimization practices while optimizing storage efficiency. This automated deletion process keeps the cloud environment organized and efficient, preventing unnecessary data accumulation.

The cloud-based server 110 is an application server or component that hosts and executes one or more backend operations, including the image extraction API 112. The server 110 is a fully managed, scalable service provided by platforms like Google Cloud Run or AWS Lambda, which enables the system to dynamically adjust resources based on the user demand in real-time. The cloud-based server's 110 role is to support the seamless and efficient processing of the scorecard images uploaded from the user-computing device 104, managing the backend processes without requiring dedicated hardware or manual server management. The server 110 operates in a serverless environment, and thus it can scale automatically, handling multiple requests simultaneously and efficiently responding to periods of high user activity without compromising performance or reliability. Another key function of this server 110 is to host the image extraction API 112, an application programming interface designed to extract the detailed data from the scorecard images uploaded by the users. For example, once an image is uploaded to cloud storage 108 and an automated trigger initiates the extraction process, the cloud-based server 110 retrieves the image and runs the image extraction API 112 on it. This API 112 is typically built using Python scripts and is enhanced by machine learning and optical character recognition (OCR) technologies. The API 112 may work in association with the advanced AI models, such as the GPT-4 Vision API, to accurately interpret and digitize the handwritten entries on the scorecard 102, converting them into structured data points. The image extraction API 112 may perform several tasks sequentially to ensure the data accuracy and completeness. For example, the API 112 may analyze the image layout to identify the sections of the scorecard (e.g., distances, shot positions, putting details), then perform OCR to read each entry and convert it into text data. If any handwriting is unclear, the API 112 may flag entries for validation, prompting the user to confirm or correct data entries. Once data extraction is complete, the API 112 may send this structured information to the application database 120 for further processing and analysis. The server's 120 infrastructure ensures that this entire process is efficient, reliable, and capable of handling a large volume of image extractions in real-time, making it an essential component of the system's backend operations.

The AI engine 114 is a component configured to interpret, analyze, and transform the raw data from the scorecard 102 into meaningful insights for the player development. The AI engine 114 leverages advanced machine learning and deep learning models to ensure that the data extracted from the scorecard 102 is accurately processed and translated into one or more actionable metrics. In an embodiment, the AI engine 114 may be configured to recognize and digitize the handwritten scorecard entries through optical character recognition (OCR), interpreting various shot metrics, and generating personalized training recommendations. This AI-powered processing may be integral to the system's ability to evaluate the one or more performance metrics, such as driving distance, fairways hit, greens in regulation, strokes gained, and putting accuracy, providing the one or more players with a comprehensive analysis of their strengths, weaknesses, and areas for improvement. Within the AI engine 114, the OCR module 116 and the image processing module (IPM) 118 may be responsible for converting the scorecard images into the structured data. The OCR module 116 uses OCR technology, specifically tailored to recognize both numerical data and handwritten text. The OCR process involves scanning the scorecard image to identify text and figures in predefined fields, such as distances to the pin, shot conditions, and putting details. To enhance recognition accuracy, the OCR module 116 may employ convolutional neural networks (CNNs) or recurrent neural networks (RNNs), both of which excel in processing and recognizing complex patterns in text and handwriting. Additionally, the OCR module 116 may prompt the user for verification if any ambiguities arise, ensuring that data inaccuracies are minimized over time as the AI model learns and adapts to player handwriting styles. The image processing module 118 may be configured to perform one or more preparatory tasks before the OCR, ensuring the image quality and orientation are optimal for analysis. Techniques such as image enhancement, noise reduction, and perspective correction may be applied to improve clarity and legibility. For example, if a scorecard image is skewed or poorly lit, the image processing module 118 adjusts the brightness, contrast, and alignment to achieve an accurate capture of the data fields. This pre-processing stage ensures that the OCR results are precise, allowing for a reliable interpretation of data.

Further, the AI engine 114 may encompass data analysis and recommendation algorithms that evaluate the player's performance metrics and generate insights. Machine learning models, trained on a vast dataset of golf statistics, apply a scoring model to compare the player's data against the benchmarked metrics, helping to identify specific areas needing improvement. For example, the AI engine 114 may analyze trends across the one or more rounds, such as a player's tendency to miss greens in regulation and recommend the targeted drills to address this weakness. The AI engine 114 may also incorporate reinforcement learning to enhance its insights and recommendations over time, continuously refining the analysis based on the historical data and the user feedback. Additionally, the AI engine 114 may support natural language generation (NLG) to translate analytical results into easily understandable summaries and personalized feedback. This feature allows the AI engine 114 to provide the one or more players with specific, actionable recommendations that encourage improvement, such as suggesting particular putting or approach shot drills. The NLG capability ensures that the one or more players can quickly grasp their performance insights, fostering greater engagement and motivation to work on their game.

The application database 120 is a robust and multi-layered storage solution that houses a wide range of player data, performance metrics, and community interactions. The database 120 has been designed with two main components: a NoSQL database and a SQL database, each optimized for specific types of data. The NoSQL database handles the unstructured or semi-structured data such as community posts, messages, user preferences, and media uploads, ensuring flexible data retrieval and rapid response times for user interactions within the mobile application 106. The SQL database stores the structured and relational data, particularly detailed scorecard metrics and performance analytics extracted from the AI engine's 114 analysis. This dual-database structure allows the application to efficiently organize and access the diverse data types, ensuring smooth operation and fast data access. The database 120 is integral to providing a personalized user experience, as it supports the retrieval of the historical performance data, facilitates player insights, and enables continuous player engagement by storing the essential records and tracking the long-term progress. Through cloud integration, the database 120 also allows for secure, scalable storage, adapting to increasing data volumes as more players engage with the system over time.

The method of operation for the golf scoring and analysis system for player development follows a process, from setting up and recording data during play to analyzing and presenting insights post-game. The first step begins with the player or scorer acquiring and preparing the physical player scorecard 102. The scorecard 102 may be specially designed with predefined sections for capturing essential scoring and performance metrics for each hole. The player or scorer fills out sections on distances to the pin, ball lies and conditions, ball locations relative to the hole, shot outcomes, and putting metrics, for ease of use and consistency. The scorecard 102 may be printed by the player or scorer or purchased as a pre-printed version to ensure uniformity across rounds. After completing the round, the next step involves using the mobile application 106 to capture the image of the filled scorecard 102. The player or scorer may open the mobile application 106 on the user-computing device 104, select the scorecard capture function, and takes a clear, complete photo of the scorecard 102 by using the device camera 104a. The mobile application 106 then automatically uploads the captured image to the cloud storage service 108a of the cloud storage device 108, which temporarily stores it and organizes it by unique identifiers, such as player ID and scorecard reference, for efficient access. This storage may serve as a temporary holding space, ensuring that the scorecard image is accessible for subsequent data extraction while preserving the original details. Once the image is stored, the cloud environment triggers the image extraction API 112, a cloud function (such as Google Cloud Run) deployed in a serverless environment to process the captured image. The API 112 may retrieve the image from storage and utilizes an AI-powered vision engine, specifically the GPT-4 Vision API, to perform optical character recognition (OCR) on the image. This step involves identifying, extracting, and digitizing handwritten entries from the scorecard 102, converting them into structured data points that can be easily analyzed. The system may prompt the player to validate entries if the handwriting is ambiguous, improving data accuracy over time as the AI refines its recognition capabilities. Following the data extraction, the structured data is transferred to the application database 120, which consists of both the NoSQL and SQL database. The NoSQL database may be used for general information, such as community posts, messages, and user preferences, while the SQL database stores structured scorecard data like scores, shot details, and performance metrics. This database structure supports efficient data retrieval, enabling detailed performance analysis while maintaining user-specific information and media in organized repositories. With the data now stored in the database 120, the AI engine 114 may be configured to initiate the analysis phase, applying machine learning models trained on historical golf data to provide an in-depth performance review. The AI engine 120 interprets key metrics such as driving distance, fairways hit, greens in regulation, strokes gained across different aspects of play, and putting performance indicators. Based on this analysis, the system identifies specific strengths, weaknesses, improvement opportunities, and threats to the player's game. Additionally, it generates the personalized insights and recommendations for training drills and skill refinement tailored to the player's current performance profile. The process further involves displaying the analyzed data and insights in the charting and visualization solution, which is accessible through the mobile application 106. Using tools such as Tableau, the system presents data in interactive charts, visualizations, and summary reports, allowing the player to explore their performance metrics in a user-friendly format. The player may view specific metrics from their most recent game, identify trends across multiple rounds, and access targeted training recommendations to support ongoing development. This visual data interaction reinforces engagement, motivating players to make actionable improvements and track their progress over time. By following the above steps, the golf scoring and analysis system offers an integrated, seamless solution for capturing, analyzing, and applying performance data, delivering comprehensive insights into a player's strengths, weaknesses, and potential areas for growth.

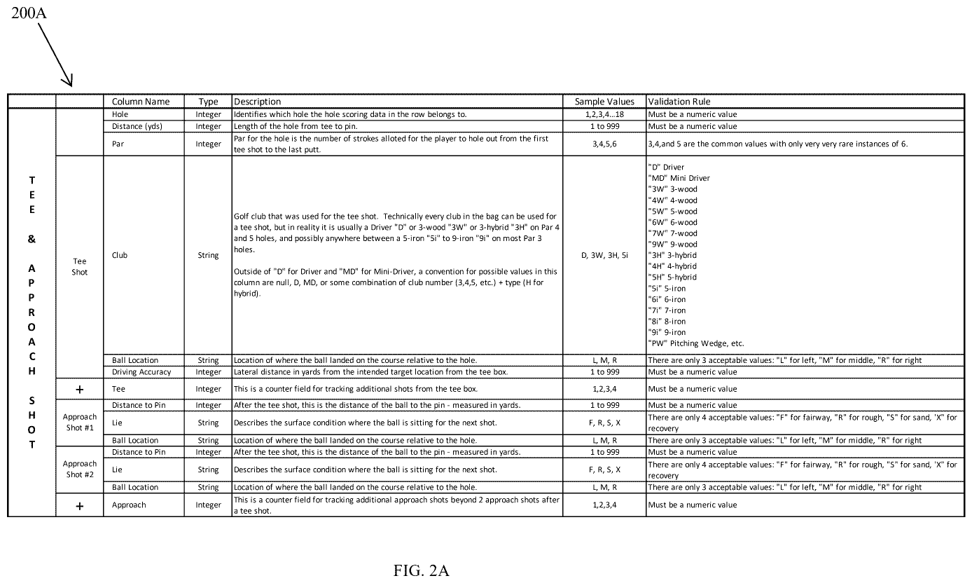

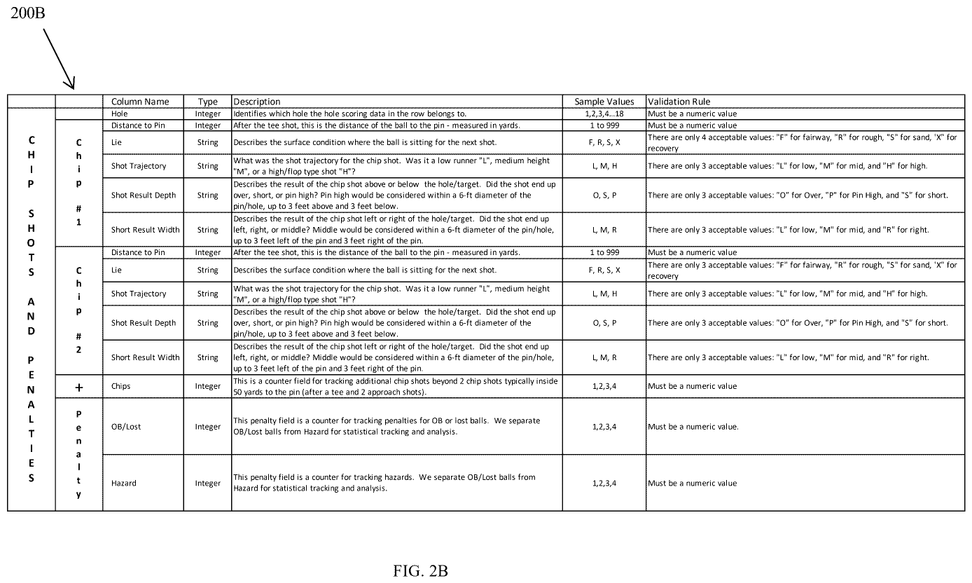

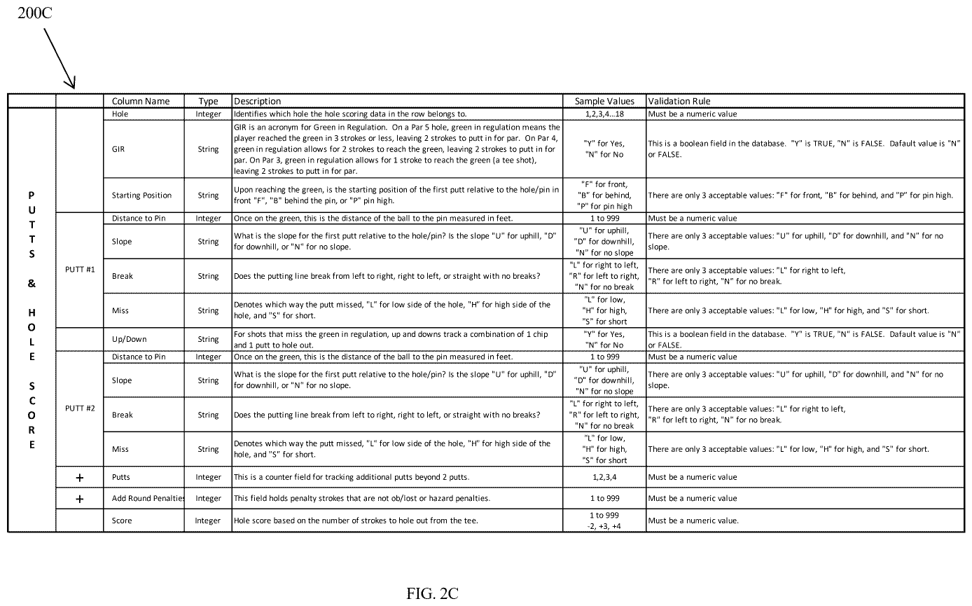

FIG. 2 is a diagram 200 that illustrate a table defining key information related to golf and its scoring system, in accordance with an embodiment of the present invention. The table has been designed for capturing detailed golf performance metrics on the scorecard 102, covering tee shots, approach shots, chip shots, penalties, putts, and hole scores. Each section contains columns for data input fields, specified data types, descriptions, sample values, and validation rules, ensuring accurate and consistent data capture.

In the Tee and Approach Shot section, it begins with the Hole column, identifying the hole number as an integer (1-18). Distance (Yds) records the length of the hole from tee to pin as an integer between 1 and 999. Par captures the expected strokes to complete the hole, usually 3, 4, or 5. Club specifies the golf club used for the tee shot, allowing values like “D” for Driver, “3W” for 3-wood, or “9i” for 9-iron. Ball Location indicates where the ball lands (L for Left, M for Middle, R for Right), while Distance to Pin records the ball's distance to the pin post-tee shot. Lie describes the surface condition (F for fairway, R for rough, S for sand, X for recovery). The Approach Tally Mark tracks extra approach shots using tally marks or integers. GIR (Green in Regulation) indicates whether the player reached the green within the regulated strokes for par, marked with a checkmark or null.

The Chip Shots and Penalties section includes Chip #1 and Chip #2, which record details like shot trajectory (L for low, M for mid, H for high), result depth (O for Over, S for Short, P for Pin High), and width (L for left, M for middle, R for right). The +Chips column counts extra chip shots beyond two, using tally marks or integers. Penalty OB/Lost captures penalties for out-of-bounds or lost balls with tally marks or numbers, denoting a stroke plus distance penalty. Penalty Hazard is similar but specific to hazards, resulting in a 1-stroke penalty. Penalty Strokes provides a field for additional penalties, with tally marks or numbers as input options.

The Putts and Hole Score section includes PUTT #1 and PUTT #2 fields for tracking putting details, such as the starting position (F for front, B for behind, P for pin high), distance to the pin in feet, slope (U for uphill, D for downhill, N for no slope), break direction (L for right to left, R for left to right, N for no break), and miss (L for low, H for high, S for short). Up/Down marks successful up-and-down attempts with a checkmark. The +Putts field records additional putts beyond two using tally marks or integers. The Score column provides the hole score as the number of strokes to hole out, while Stroke Differential records the difference between the player's score and par, using signed integers like +1, −1, or 0.

Each entry has strict validation rules to ensure data integrity, with acceptable values specified for each field. This structured approach allows for comprehensive performance tracking, enabling detailed analysis and insights into each aspect of the player's game. The detailed layout has been designed to facilitate consistent and accurate data capture, supporting both user experience and backend data processing in the golf scoring system.

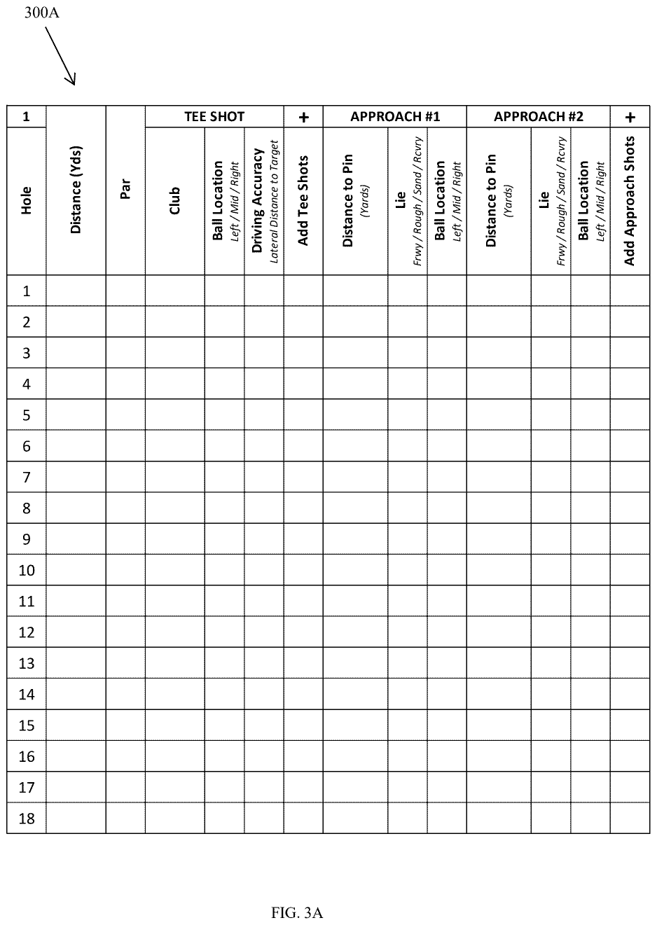

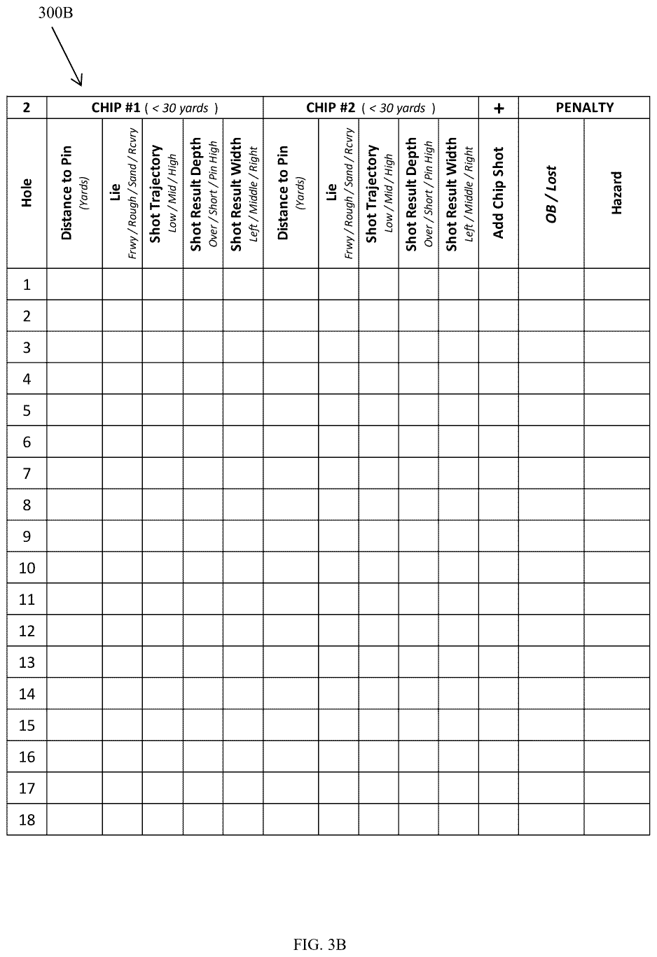

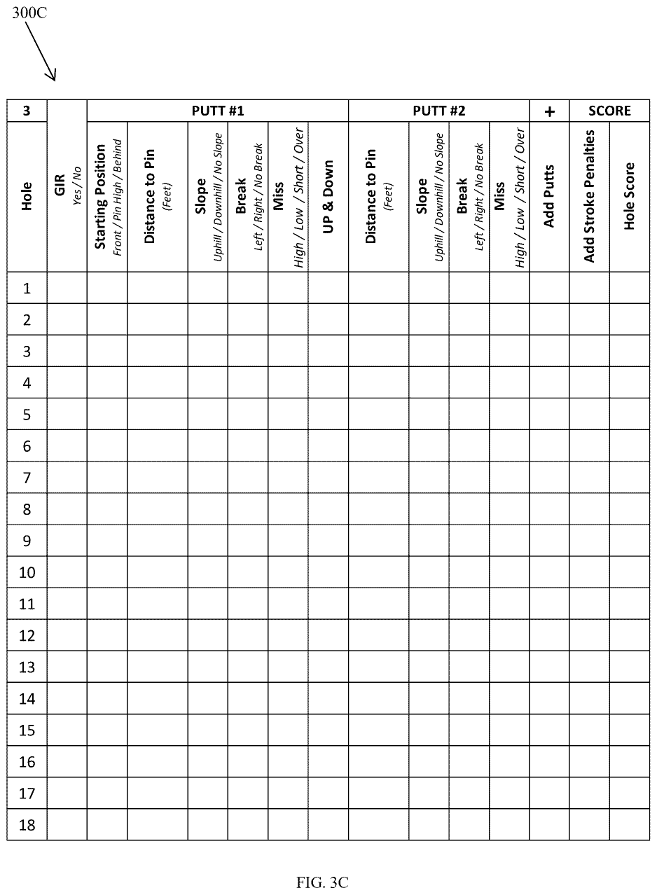

FIGS. 3a-3c are diagrams 300a-300c that illustrate exemplary scorecards, in accordance with an embodiment of the present invention. The tables 300a-300c provides detailed templates for three exemplary golf scorecards, each designed to capture essential data for analyzing player performance comprehensively. These scorecard templates aim to facilitate organized and structured data entry during a round of golf, enabling the players or scorers to record every relevant metric necessary for a detailed post-round analysis. Each scorecard template covers different aspects of play and includes fields for capturing tee shots, approach shots, chip shots, putts, and penalties. The layouts are arranged to simplify recording, with clearly labeled columns and rows for each type of shot and specific performance metric. The key fields across the templates include Hole Number, Par, Distance to Pin, and Lie (indicating the surface condition), allowing the users to record data for each hole systematically. Additionally, club selection is recorded on these scorecards, providing insights into a player's club usage and decision-making process for each shot. The templates also emphasize the capture of chip shot details and penalty tracking. The specific fields allow the users to record shot trajectory, shot result depth (indicating whether the shot was over, short, or pin high), and result width (whether the shot was left, right, or center). These sections are particularly useful for tracking shot accuracy and the player's ability to position the ball relative to the hole. Penalties are recorded in dedicated sections, where players can note penalties for out-of-bounds shots, lost balls, or hazard infractions. Each template includes a tally system or numerical input for easy penalty tracking. The putting section of each scorecard template has been designed to capture the subtleties of putting performance, including starting position relative to the hole, distance to pin, slope, break direction, and whether the putt missed high, low, or short. This detailed data collection allows for granular analysis of putting accuracy and the player's ability to read greens. The templates also include fields for calculating Score and Stroke Differential, allowing a quick assessment of performance against par for each hole.

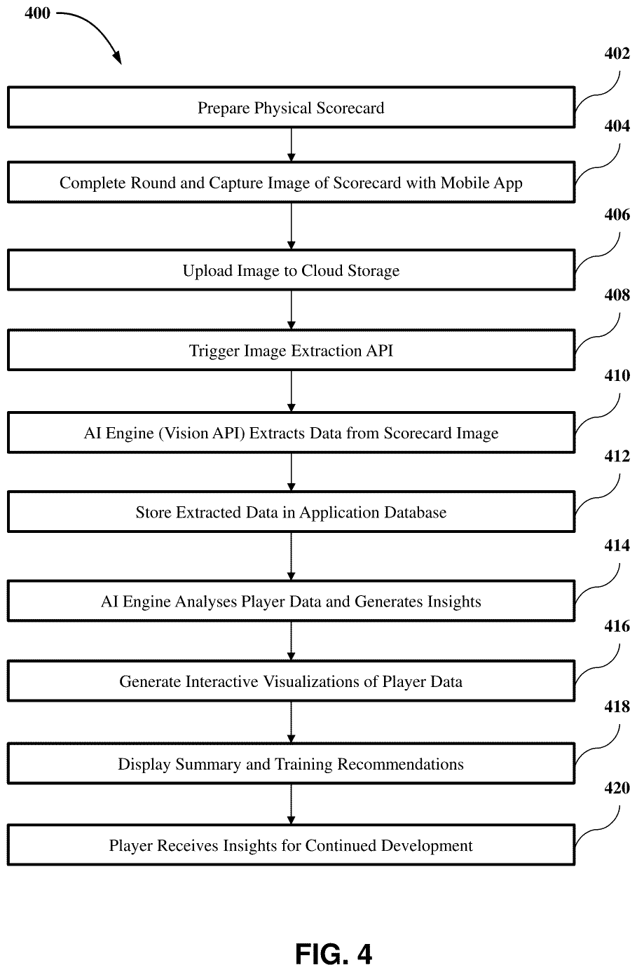

FIG. 4 is a diagram 400 that illustrate a flowchart for the golf scoring and analysis system's method operation, in accordance with an embodiment of the present invention.

At step 402, the player or scorer obtains the physical player scorecard 102, which is designed with specific sections for recording the hole-by-hole data. The player or scorer fills out details, such as distances to the pin, shot conditions, and ball positions, for each hole as the player progresses through the round. This structured data capture allows for consistent and accurate recording of key performance metrics in a familiar format.

At step 404, once the round is completed, the player or scorer opens the mobile application 106 on the user-computing device 104 and selects the option to capture a photo of the filled scorecard 102. This image capturing step is essential, as it initiates the process of transferring the handwritten data into the digital format. The mobile application 106 prompts the player or scorer to take a clear, high-resolution photo of the scorecard 102, ensuring that all data entries are fully visible and readable.

At step 406, after capturing the image, the mobile application 106 automatically uploads it to the cloud storage device 108. The cloud storage service 108a of the cloud storage device 108 may organize each image using unique identifiers, such as player ID and scorecard reference. This temporary storage serves as a holding space, ensuring that the image remains accessible and organized for the subsequent data extraction step.

At step 408, once the image is uploaded to the cloud storage device 108, the system triggers the image extraction API 112 hosted on the cloud-based server 110. This cloud function retrieves the scorecard image from the storage and prepares it for data extraction. The image extraction API 112 may be deployed in a serverless environment like Google Cloud Run and ensure that the system can dynamically scale to handle multiple users'scorecard images simultaneously, providing a robust infrastructure for processing.

At step 410, the image extraction API 112 calls the AI-powered Vision API (AI engine 114), specifically designed for OCR and image processing. The AI engine 114 may identify and interpret each entry on the scorecard 102, extracting essential data points such as shot distances, ball lies, and putting details. The OCR engine 116 may prompt the player to verify unclear entries, increasing accuracy over time as the AI model learns from the player's inputs.

At step 412, after the data extraction, the structured data may be transferred to the application database 120, which comprises both the NoSQL and SQL databases. The NoSQL database manages general information, such as community interactions and user preferences, while the SQL database organizes the scorecard data for structured retrieval and analysis. This separation ensures efficient data management and optimizes storage for different types of user information.

At step 414, the AI engine 114 then proceeds to analyze the extracted data, applying machine learning algorithms to interpret performance metrics like driving distance, fairways hit, greens in regulation, strokes gained, and putting accuracy. The AI engine 114 plays a critical role in transforming the raw performance data into meaningful insights by applying advanced machine learning algorithms to evaluate various metrics and identify areas for player improvement. Once the data from the scorecard is extracted and organized, the AI engine 114 begins by analyzing core performance metrics such as driving distance, fairways hit, greens in regulation, strokes gained in different aspects of play (off the tee, approach, around the green, and putting), and putting accuracy. Each metric represents a fundamental component of a golfer's performance, providing a foundation for understanding their overall game and pinpointing specific strengths and weaknesses. To interpret these metrics, the AI engine 114 may use a scoring model, which may compare the player's data against benchmarked performance standards or historical data for that player. For example, if the player consistently misses fairways or greens, the AI engine 114 will identify this as an area for improvement. By analyzing patterns within the data, the AI engine 114 can recognize trends such as recurring errors or consistent strengths, helping to highlight areas where the player excels and those that require attention. For example, if the AI engine 114 identifies that the player struggles with accuracy on approach shots, it may suggest drills that target precision or shot placement. Based on this analysis, the AI engine 114 may generate the personalized training recommendations, which are tailored to address the identified weaknesses or reinforce existing strengths. These recommendations may include specific drills, exercises, or practice routines designed to improve particular aspects of the player's game, such as putting drills to enhance accuracy or driving exercises to increase distance. The training guidance is aimed at fostering targeted improvements and offers a structured path for the player to focus on critical areas in subsequent practice sessions. This customized feedback not only supports continuous player development but also promotes engagement by providing actionable, goal-oriented suggestions that align with the player's current needs and performance level.

At step 416, the analyzed data is then presented to the player through the charting and visualization solution, accessible within the mobile application 106. Interactive visualizations, created using tools like Tableau, may allow the players to explore their performance metrics in detail. They can view recent game statistics, observe trends over multiple rounds, and review personalized suggestions for practice drills to enhance specific skills.

At step 418, the mobile application 106 displays the summary report of the round, highlighting key metrics and areas for improvement. The application 106 provides targeted recommendations, encouraging players to focus on identified weaknesses and capitalize on their strengths through guided practice and specific drills.

At step 420, the player will have access to the comprehensive insights and actionable feedback, empowering them to make informed improvements to their game. The system's end-to-end data capture, analysis, and feedback mechanism ensures an efficient and user-friendly experience, supporting long-term player development and engagement with the platform.

The foregoing descriptions of specific embodiments of the present technology have been presented for purposes of illustration and description. They are not intended to be exhaustive or to limit the present technology to the precise forms disclosed, and obviously many modifications and variations are possible considering the above teaching. The embodiments were chosen and described to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology. While several possible embodiments of the invention have been described above and illustrated in some cases, it should be interpreted and understood as to have been presented only by way of illustration and example, but not by limitation. Thus, the breadth and scope of a preferred embodiment should not be limited by any of the above-described exemplary embodiments.

1. A golf scoring and analysis system for player development, the system comprising:
a physical player scorecard configured to record hole-by-hole scoring data and key metrics related to a player's round of golf, the scorecard including sections for capturing information about distances, ball lies and conditions, shot outcomes, and putting characteristics;
a mobile application executable on a user computing device, the mobile application configured to capture an image of the scorecard and transmit the image for data extraction and analysis;
a cloud storage service configured to temporarily store the captured image of the scorecard;
an image extraction API, deployed on a cloud-based server, configured to receive the image from the cloud storage service and call an AI engine to extract scorecard data from the image;
the AI engine, comprising an OCR and image processing module, configured to:
process the image by performing at least one of an image enhancement, a noise reduction, and a perspective correction;
analyze the processed image to identify one or more sections of the scoreboard;
convert the extracted scorecard data using the analysis into a structured format within an application database; and
extract user notes in the scorecard to store in a NoSQL database; and
the application database, comprising the NOSQL database for general data that comprises the user notes and the SQL database for the extracted scorecard data, the database configured to store player scoring data, analytics, and community interactions,
wherein the AI engine is further configured to:
analyse the scorecard data using a scoring model comprising one or more machine learning algorithms trained on historical performance data, the scoring model being applied to compute performance metrics, including driving distance, fairways hit, greens in regulation, strokes gained, and putting accuracy; and
generate personalized insights and recommendations for training drills and skill improvement based on the computed performance metrics and the extracted scorecard data, the recommendations comprising drills, exercises, and practice routines tailored to identified weaknesses or strengths, and
wherein the mobile application is further configured to present analytics and performance metrics to the player in an interactive manner and display the personalized insights and recommendations.

2. The system of claim 1, wherein the scorecard is configured to be printed or purchased by the player prior to play and includes pre-defined sections for recording data inducing at least distances to pin, ball location, shot flight trajectory, and putting characteristics for each hole.

3. The system of claim 2, wherein the scorecard further includes an area for capturing the user notes or observations about the round, allowing for additional context to be added to the scorecard data.

4. The system of claim 1, wherein the mobile application is further configured to prompt the player to validate the extracted data based on OCR accuracy and legibility of handwriting on the scorecard.

5. The system of claim 1, wherein the cloud storage service is further configured to organize scorecard images by unique identifiers corresponding to the player and scorecard reference, allowing for efficient retrieval and temporary storage prior to the data extraction.

6. The system of claim 1, wherein the image extraction API is implemented as a Python script deployed within a serverless computing environment to process the scorecard image and parse the extracted data into the application database.

7. The system of claim 1, wherein the AI engine includes a generative pre-trained transformer (GPT) vision API configured to extract the scoring data from the scorecard image by identifying and interpreting each data entry location on the scorecard.

8. The system of claim 1, wherein the application database utilizes the NOSQL database for storing community posts, messages, and user preferences, and the SQL database for storing the structured scorecard data, each configured for optimized data retrieval and processing.

9. The system of claim 1, wherein the mobile application includes a charting and visualization solution that is integrated with a third-party visualization tool and is configured to enable the player to interact with the performance metrics and view analytical reports generated from the scorecard data.

10. The system of claim 1, wherein the charting and visualization solution is configured to display metrics such as average driving distance, fairways hit, greens in regulation, strokes gained in different aspects of play, and putting performance indicators.

11. The system of claim 1, wherein the AI engine further includes a machine learning model trained on historical golf scoring data to provide accurate and personalized insights and recommendations for the player's training and skill improvement.

12. The system of claim 11, wherein the AI engine is further configured to identify specific player strengths, weaknesses, and improvement opportunities based on the analyzed data and present this information to the player via the mobile application's user interface.

13. The system of claim wherein the mobile application further includes a user interface for accessing drill practice logs, viewing historical performance metrics, and interacting with a community of other users.

14. The system of claim 13, wherein the mobile application is further configured to allow the player to upload video or image files for review alongside the scoring data, storing these in the application database for comprehensive performance tracking.

15. The system of claim 1, wherein the AI engine is further configured to provide trend analysis of the player's performance over multiple rounds, identifying patterns and long-term improvements or areas requiring additional focus.

16. The system of claim 1, wherein the cloud storage service is further configured to automatically delete the image of the scorecard from storage once the data extraction has been completed and verified, maintaining player privacy and storage efficiency.

17. The system of claim 1, wherein the mobile application is further configured to display a summary report after each round, highlighting key performance metrics and suggesting specific drills or practice routines tailored to the player's recent performance.

18. The system of claim 1, wherein the application database includes a scoring history log that allows the player to access, review, and analyze data from previous rounds for tracking long-term progress.

19. A golf training system comprising:
a scorecard including fields for recording metrics regarding player performance by hand wherein the fields are arranged to facilitate accurate optical character recognition;
an application adapted to be stored on and execute on a mobile device wherein the application £ includes instructions to:
scan the scorecard using a camera of the mobile device;
capture the metrics in the scan; and
generate a training plan for the player based on the captured metrics.
